(19) United States
(12) Patent Application Publication
(10) Pub. No.: US 2026/0285947 A1
(43) Pub. Date: Sep. 24, 2026

(54) ANTIBODY PRODUCTION

(71) Applicant: ARIEL SCIENTIFIC INNOVATIONS LTD., Ariel (IL)

(72) Inventors: Asher YAHALOM, Givat Shmuel (IL); Michael A FIRER, Karnei Shomron (IL); Ayan BARBORA, Ariel (IL)

(21) Appl. No.: 19/476,731

(22) PCT Filed: May 8, 2024
(86) PCT No.: PCT/IB2024/054486

§ 371 Date: Oct. 20, 2025

Related U.S. Application Data
(60) Provisional application No. 63/464,655, filed on May 8, 2023.

(51) Int. Cl. C07K 16/00; C12M 1/00; C12M 1/42; C12M 3/00; C12N 13/00

(52) U.S. Cl. CPC C07K 16/00; C12M 23/44; C12M 35/02; C12M 41/14; C12N 13/00

(57) ABSTRACT

Disclosed are methods and devices that are suitable for producing monoclonal antibodies. In some embodiments, the method comprises: i. providing antibody-producing cells; ii. incubating the cells in vitro in the presence of an antigen for an incubation period under conditions suitable for the cells to produce antibodies; and iii. during, before or after at least some of said incubation period, irradiating the cells with millimeter wave (MMW) and/or terahertz radiation for an irradiation period, thereby the cells producing antibodies. In some embodiments, the device comprises: an incubation module suitable for incubation of antibody-producing cells for an incubation period therein; and an irradiation module for irradiation of antibody-producing cells that are being incubated in the incubation module components for an irradiation period with MMW and/or terahertz radiation.

### RELATED APPLICATION

The present application gains priority from U.S. Provisional Patent Application 63/464,655 filed 8 May 2023 which is included by reference as if fully set-forth herein.

### FIELD AND BACKGROUND OF THE INVENTION

The invention, in some embodiments, relates to the field of antibodies and, more particularly but not exclusively, to methods and devices that are suitable for producing antibodies.

Monoclonal antibodies (mAb) are a major product of the biotechnological industry, being used in research, diagnostics, therapeutics, and industrial processing. mAb therapeutics for research use and clinical applications constitute a major sector of the biotech industry. Following intense efforts in molecular and structural biology and immunology since the year 2000 an increasing number of mAb are used as therapeutics, driving competition to produce antibodies at reasonable costs without compromising quality.

Viruses are known pathogens that mutate rapidly making it challenging to develop treatments and vaccines. One accepted treatment for a viral infection is convalescent plasma therapy (CPT). The utility of CPT may be limited by the number of recovered people who are able to donate plasma.

There is a need for increasing the efficiency and/or output of mAb production, for example for various uses, including research, diagnostics and therapy.

### SUMMARY OF THE INVENTION

Some embodiments of the invention herein provide methods and devices that are suitable for antibody production. In some embodiments, the methods and devices increase antibody production from cells grown in vitro, for example, in bioreactors.

According to an aspect of some embodiments of the teachings herein there is provided a method for producing monoclonal antibodies, comprising:
i. providing antibody-producing cells;
ii. incubating the cells in vitro in the presence of an antigen for an incubation period under conditions suitable for the cells to produce antibodies; and
iii. during, before or after at least some of the incubation period, irradiating the cells with MMW and/or terahertz radiation for an irradiation period, thereby the cells producing antibodies.

As a result of irradiating the cells, the yield of antibodies is higher than under the same incubation conditions without the irradiating.

In preferred embodiments, the method further comprises collecting antibodies produced by the cells. In some embodiments, the collecting of the antibodies is subsequent to the incubation period. In some alternative embodiments, at least some of the collecting of the antibodies is during the incubation period.

### Suitable Cells

Any suitable immune cell that produces antibodies may be used for implementing the teachings herein.

In some embodiments, the cells are IgA-producing cells. The inventors found that under conditions according to the teachings herein, exemplary IgA producing cells (MOPC315.BM cells) produced about a 24× yield of IgA antibodies compared to the same incubation conditions without irradiation.

In some embodiments, the cells are IgG-producing cells. The inventors found that under conditions according to the teachings herein, exemplary IgG producing cells (U13.6 cells) produced about a 1.4× yield of IgG antibodies compared to the same incubation conditions without irradiation.

In some embodiments, the antibody-producing cells are selected from the group consisting of CHO cells, HEK cells and hybridoma cells.

In some embodiments, the antibody-producing cells are immune cells.

### Incubation Conditions

The antibody-producing cells are incubated in vitro in the presence of an antigen for an incubation period under conditions suitable for the cells to produce antibodies. The conditions of such incubation are standard incubation conditions as known in the art for any particular type of cell, including incubation temperature, medium, antigen, reagent concentration and nutrient concentration, cell concentration and the like. A person having ordinary skill in the art implementing the teachings herein knows to consult standard references to find incubation conditions for any particular type of antibody-producing cell.

In some embodiments, substantially all of the cells (not less than 90%, and even not less than 95%) that are found in a single incubation vessel are irradiated at the same time during the irradiation period. Alternatively, in some embodiments, at any given moment during the irradiation period, only some cells found in a single incubation vessel are irradiated at the same time.

### Timing of Irradiation Period

During, before or after at least some of the incubation period the cells are irradiated for an irradiation period

In some embodiments, at least some of the irradiation period is during the incubation period so that for at least some time irradiating and incubating is performed simultaneously. Typically, when irradiating and incubation are performed simultaneously, the irradiation is performed at the incubation temperature.

In some embodiments, the irradiation period begins prior to the incubation period and continues during at least some of the incubation period.

In some such embodiments, the irradiation period begins simultaneously with or during the incubation period and continues during at least some of the incubation period. In some such embodiments, the irradiation period ends prior to the end of the incubation period. Alternatively, in some such embodiments the irradiation periods simultaneously with or subsequent to the end of the incubation period.

In some embodiments, all of the irradiation period is prior to the incubation period, that is to say, the irradiation period begins and ends prior to the beginning of the incubation period. In such embodiments, irradiation is performed at any suitable temperature, for example, at room temperature but in some embodiments at the incubation temperature.

### Duration of Irradiation Period

The duration of the irradiation period is any suitable duration.

In some preferred embodiments, the duration of irradiation period is not less than about 5 minutes and even not less than about 10 minutes. In the experimental section, the Inventors tested irradiation periods having a duration of about 15 minutes.

In some embodiments, especially in some embodiments where the irradiation period ends prior to the incubation period, the duration of the irradiation period is not more than about 120 minutes and even not more than about 60 minutes.

In some embodiments, especially in some embodiments where at least some of the irradiation period is during the incubation period, the duration of the irradiation period is longer than 60 minutes and even longer than 120 minutes. For example, in some such embodiments, the duration of the irradiation period is not less than 5%, not less than 10% and even not less than 20% of the incubation period.

In some embodiments, the irradiation period is a single contiguous period of time. In some alternative embodiments, the irradiation period comprises at least two time-segments, any two time segments separated by a rest period, wherein a sum of the durations of all of the time segments is the duration of the irradiation period.

### Frequency of Radiation

The cells are irradiated with any suitable frequency or frequencies of MMW and/or terahertz radiation.

In some embodiments, the radiation comprises MMW radiation having one or more frequencies of between about 30 GHz and about 300 GHz. Additionally or alternatively, in some embodiments, the radiation comprises terahertz radiation having one or more frequencies of between about 0.1 THz (100 GHz) and about 10 THz (10000 GHz).

In some embodiments, the radiation consists of terahertz radiation having one or more frequencies of between about 0.1 THz and about 10 THz.

In some embodiments, the radiation consists of MMW radiation having one or more frequencies of between about 30 GHz and about 300 GHz.

In some embodiments, the radiation comprises, and in some embodiments consists of, MMW radiation having one or more frequencies of between about 65 GHz and about 120 GHz.

In some embodiments, the radiation comprises, and in some embodiments consists of, MMW radiation having one or more frequencies of between about 70 GHz and about 115 GHz.

In some embodiments, the radiation comprises, and in some embodiments consists of, MMW radiation having one or more frequencies of between about 75 GHz and about 110 GHz.

In some embodiments, the radiation comprises, and in some embodiments consists of, MMW radiation having one or more frequencies of between about 85 GHz and about 105 GHz.

In some embodiments, the radiation comprises, and in some embodiments consists of, MMW radiation having one or more frequencies of between about 70 GHz and about 110 GHz.

Irradiation with a Single Frequency

In some embodiments, during at least some of the irradiation period, the irradiation is with a single frequency of radiation at any one moment. In some such embodiments, during the entire irradiation period, the irradiation is with a single frequency of radiation at any one moment.

In some such embodiments, during the irradiation period, the frequency of the single frequency of radiation is fixed and does not change.

Alternatively, in some such embodiments, during the irradiation period, the single frequency of the radiation changes, that is to say that at any given moment the cells are irradiated with only a single frequency but the specific single frequency that irradiates the cells at any given moment changes during the irradiation period. In some embodiments, the irradiation comprises frequency sweeping during at least some of the irradiation period so that at any given moment the cells are irradiated with only a single frequency but the specific single frequency that irradiates the cells at any given moment changes monotonously during the irradiation period. In some such embodiments, monotonously changing is exclusively increasing frequency, preferably at a substantially constant rate of increase in frequency/unit time, in some embodiments a continuously increasing and in some embodiments increasing step-wise. In some such embodiments, monotonously changing is a exclusively decreasing frequency, preferably at a substantially constant rate of decrease in frequency/unit time, in some embodiments continuously decreasing and in some embodiments decreasing step-wise.

In the Experimental section, the Inventors describe irradiation by sweeping a frequency range during the irradiation period so that at any given moment the cells are irradiated with only a single frequency but the specific single frequency that irradiated the cells at any given moment changed step-wise during the irradiation period, from 85 GHz to 110 GHz in 200 steps.

Irradiation with Multiple Frequencies

In some embodiments, during at least some of the irradiation period, the irradiating is with multiple frequencies of radiation simultaneously. In some such embodiments, during the entire irradiation period the irradiating is with multiple frequencies of radiation simultaneously.

In some such embodiments, during the irradiation period, the specific combination of frequencies of the multiple frequencies of radiation is fixed and does not change.

Alternatively, in some such embodiments, during the irradiation period, the specific combination of frequencies of the radiation changes.

### Intensity of Irradiation

The intensity of the irradiation is any suitable intensity.

In some embodiments, during the irradiation period, the irradiating has an average intensity of between about 10 W/m² and about 600 W/m², an average intensity of between about 50 W/m² and about 400 W/m², an average intensity of between about 100 W/m² and about 200 W/m², and even between about 110 W/m² and about 170 W/m², for example, between about 116.67 W/m² and about 166.67 W/m². That said, in some preferred embodiments, during the irradiation period, the intensity of the irradiating does not exceed an intensity of about 200 W/m².

In some embodiments, during the irradiation period the intensity of irradiation is constant, e.g., is within about 33% of the average intensity value of the irradiation period, within about 25% of an average intensity value and even within about 15% of an average intensity value. In some alternative embodiments, the intensity of the irradiating varies by more than about 33% of an average intensity value.

The teachings herein can be implemented using a combination of known devices, for example, as described in the experimental section. That said, in some instances, the teachings herein are preferably implemented using a device according to the teachings herein that combines both an incubating and an irradiation functionality.

According to an aspect of some embodiments of the teachings herein, there is also provided a device suitable for producing antibodies comprising:
an incubation module suitable for incubation of antibody-producing cells for an incubation period therein; and
an irradiation module for irradiation of antibody-producing cells that are being incubated in the incubation module for an irradiation period with MMW and/or terahertz radiation.

In some embodiments, the irradiation module is configured for irradiation of antibody-producing cells that are being incubated in the incubation module for an irradiation period with MMW. Additionally or alternatively, in some embodiments, the irradiation module is configured for irradiation of antibody-producing cells that are being incubated in the incubation module for an irradiation period with terahertz radiation.

In some preferred embodiments, a specific irradiation module is configured to implement one, some or all of the embodiments of the method according to the teachings herein. The various options are understood by a person having ordinary skill in the art and are not repeated here for the sake of brevity

### Incubating Module

The incubating module is any suitable component of combination of components suitable for incubating antibody-producing cells as known in the art. Typically, an incubating module includes at least one temperature-controlling component configured to maintain the temperature of the contents of an incubation vessel at a desired incubation temperature.

As is known in the art, during incubation antibody-producing cells are held in an incubation vessel in the presence of a medium, the medium comprising an antigen and a growth medium that includes, inter alia, nutrients and reagents. The teachings herein are implemented using any suitable incubation vessel, including, but not limited to, a bioreactor vessel, a (Eppendorf) vial, a test tube, a dish and a microplate. In some embodiments, an incubation vessel is a multiple use vessel, for example, a stainless steel bioreactor. Alternatively, in some embodiments an incubation vessel is configured for single use, for example, a disposable bioreactor of polymer as known in the art.

In some embodiments, an incubating module comprises at least one incubation vessel for holding the antibody-producing cells in growth medium during the incubation period.

In some embodiments, an incubating module comprises at least one incubation vessel-holder for holding an incubation vessel that is not a component of the device during the incubation period.

### Irradiation Module

A device according to the teachings herein comprises an irradiation module for irradiation of antibody-producing cells that are being incubated in the incubation module for an irradiation period with MMW and/or terahertz radiation.

The irradiation module typically includes at least one radiation source (for generating MMW and/or terahertz radiation), at least one power supply for providing power to the radiation source, optionally at least one tuner for selecting and/or changing the wavelength or wavelengths used for irradiation and optionally at least one waveguide to direct generated radiation from the radiation source towards antibody-producing cells that are being incubated in the incubation module

### Controller

In preferred embodiments, the device comprises a control comprising a computer with a computer memory that is configured (e.g., with software, firmware, and/or hardware) to control the incubation module, the irradiation module or both. Such a controller can be configured to activate the device components to implement, preferably automatically, embodiments of the method according to the teachings herein, for example, various embodiments of the irradiation as described above.

In some embodiments, the incubation module comprises an own controller, e.g., to control incubation temperature, incubation duration, shaking/stirring and the like. In preferred embodiments, the incubation module is at least partially and more preferably complete, controlled by the controller of the device.

In some embodiments, the irradiation module comprises an own controller, e.g., to control irradiation frequency, irradiation duration, irradiation intensity and the like. In preferred embodiments, the irradiation module is at least partially and more preferably complete, controlled by the controller of the device.

In some embodiments, the irradiation module is configured to irradiate cells when these are not necessarily being incubated. For example, such a device comprises two stations, an irradiation station constituting the irradiation module and an incubation station constituting the incubation module. Preferably, such a device further comprises a transporter (e.g., a robot, a transport belt) for moving a cell container holding cells between the two stations. Such an embodiment is schematically depicted in FIG. 1, device 10. Device 10 comprises a controller 12 (a software and hardware configured general-purpose computer), an incubation station 14 including an incubation module 16, an irradiation station 18 including an irradiation module 20, as a transporter a conveyor belt 22 that moves a covered microplate 24 (that contain antibody-producing cells in medium) between incubation station 14 and irradiation station 18. Incubation module 16 is substantially similar to commercially-available microplate incubators modified to allow automated placement and removal of a microplate from the incubator using conveyor belt 22. Irradiation module 20 comprises a radiation source 26, a power supply 28, a tuner 30 and a waveguide 32 to direct generated MMW and/or terahertz radiation from radiation source 26 towards microplate 24 held in holder 34 of conveyor belt 22. All components of device 10 are controller by controller 12 including when a microplate is moved between stations 14 and 18, irradiation parameters at irradiation station 18 and incubation parameters at incubation station 14.

In some embodiments of a device according to the teachings herein, the irradiation module is configured to irradiate cells that are in the incubation module, optionally during the incubation of the in the incubation module. For example, in some embodiments such a device comprises a waveguide that directs generated radiation at a cell container holding cells when the cell container is being incubated in the incubation module. Such an embodiment is schematically depicted in FIG. 2, device 36. Device 36 is substantially an incubation module 16 and an irradiation module 20 as described above for device 10, where waveguide 32 is configured to direct radiation generated by radiation source 26 towards a microplate 24 that is being incubated in incubation module 16.

In some embodiments, such as in device 10 depicted in FIG. 1 and device 36 depicted in FIG. 2, all the cells are irradiated at the same time.

In some alternative embodiments, only some of the cells that are being incubated in an incubation vessel are irradiated at any one time.

A first such embodiment is schematically depicted in FIG. 3, device 38. In device 38, incubation module 16 comprises a bioreactor 40 as an incubation vessel and includes a heater/cooler 42 and a stirrer 44. Irradiation module 20 is as describe above for device 36 and is configured to directly irradiate contents 46 of bioreactor 40 inside bioreactor 40 through a window 48 that is transparent to the radiation produced by radiation source 26. Due to the large size of bioreactor 40, irradiation module 20 can only irradiate a portion of contents 46 of bioreactor 40 at one time. However, due to the stirring of contents 46 of bioreactor 40 by stirrer 44, during incubation all of the contents 46 of bioreactor 40 are irradiated.

A second such embodiment is schematically depicted in FIG. 4, device 50. In device 50, incubation module 16 comprises a bioreactor 40 as an incubation vessel and includes a heater/cooler 42 and a stirrer 44. Irradiation module 20 is as describe above for device 38. During use, pump 52 continuously (and in some embodiments, discontinuously) transfers some of contents 46 of bioreactor 40 through an irradiation vessel 54 to be irradiated by irradiation module 20 through a portion of irradiation vessel 54 that is transparent to the radiation produced by radiation source 26.

As used herein, the terms “incident power density”, “power density”, “IPD” and “PD” all refer to “power density per unit area” with units using units of W/m².

As used herein, the terms “incubation period” and “incubation period of time” are synonymous and the terms. Herein, the terms “irradiation period” and “irradiation period of time” are synonymous.

Additional aspects and embodiments of the invention are described in the specification herein below and in the appended claims.

### BRIEF DESCRIPTION OF THE FIGURES

Some embodiments of the invention are described herein with reference to the accompanying figures. The description, together with the figures, makes apparent to a person having ordinary skill in the art how some embodiments of the invention may be practiced. The figures are for the purpose of illustrative discussion and no attempt is made to show structural details of an embodiment in more detail than is necessary for a fundamental understanding of the invention. For the sake of clarity, some objects depicted in the figures are not to scale.

In the Figures:

FIG. 1 is an embodiment of a device suitable for producing monoclonal antibodies according to the teachings herein;

FIG. 2 is an embodiment of a device suitable for producing monoclonal antibodies according to the teachings herein;

FIG. 3 is an embodiment of a device suitable for producing monoclonal antibodies according to the teachings herein;

FIG. 4 is an embodiment of a device suitable for producing monoclonal antibodies according to the teachings herein;

FIG. 5A is a bar graph comparing the relative yield of antibodies produces by MOPC-IgA cells with and without irradiation according to the teachings herein;

FIG. 5B is a bar graph comparing the relative yield of antibodies produces by U13.6 IgG cells with and without irradiation according to the teachings herein;

FIG. 6A is a bar graph comparing the relative viability of MOPC-IgA cells with and without irradiation according to the teachings herein; and

FIG. 6B is a bar graph comparing the relative viability of U13.6 IgG cells with and without irradiation according to the teachings herein.

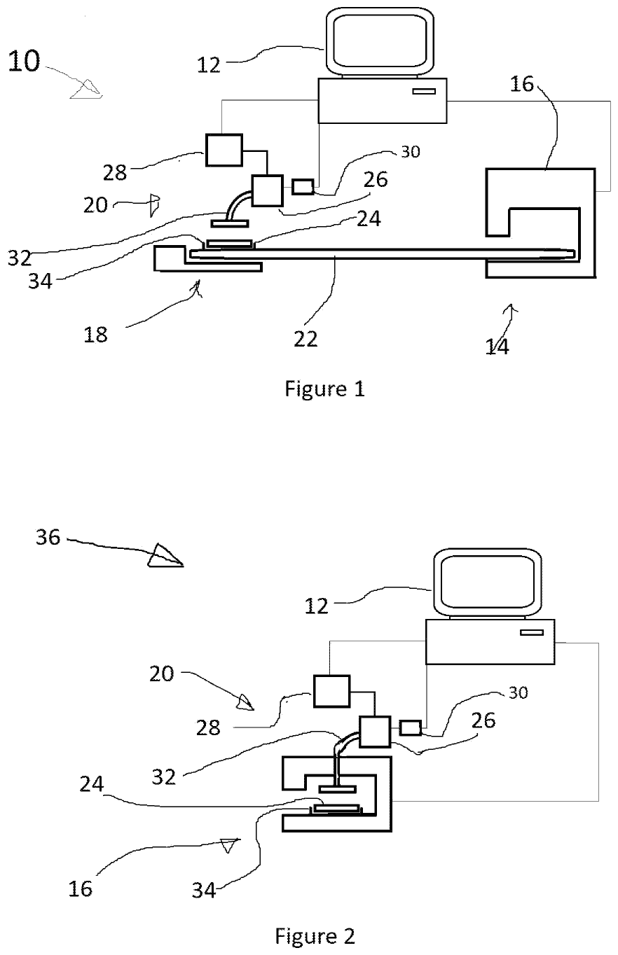

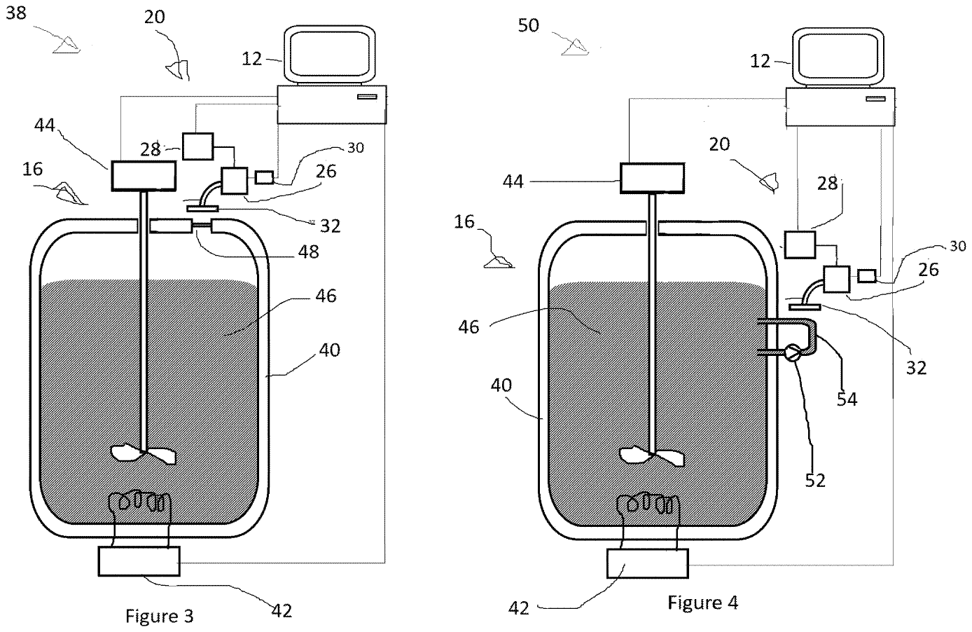

### DESCRIPTION OF SOME EMBODIMENTS OF THE INVENTION

Some embodiments of the invention herein provide methods and devices that are suitable for antibody production. In some embodiments, the methods and devices increase antibody production from cells grown in in vitro, for example, in bioreactors.

The principles, uses and implementations of the teachings of the invention may be better understood with reference to the accompanying description and figures. Upon perusal of the description and figures present herein, one skilled in the art is able to implement the teachings of the invention without undue effort or experimentation. In the figures, like reference numerals refer to like parts throughout.

Before explaining at least one embodiment of the invention in detail, it is to be understood that the invention is not necessarily limited in its application to the details of construction and the arrangement of the components and/or methods set forth herein. The invention is capable of other embodiments or of being practiced or carried out in various ways. The phraseology and terminology employed herein are for descriptive purposes and should not be regarded as limiting.

The inventions herein relate to the Inventor's discovery based on experiments showing that exemplary cells (MOPC315.BM cells [2] and U13.6 cells [3]) subjected to MMW (in the experiments using 85-105 GHz, but not necessarily limiting) sweeping radiation in vitro demonstrated enhanced antibody production without affecting cell viability.

Accordingly, the teachings herein can, in some embodiments, be used for producing a serum that can serve as a cure for a viral infection.

Lymphocytes (T-cells and B-cells) interact together in adaptive immune responses involving biochemical communication. Antigen Presenting Cells (APCs) activate naïve CD4 T cell specific for peptides from the pathogen resulting in production of armed helper T cells. B cells are the dominant APCs to activate naïve CD4+ T cells upon inoculation with a pathogen. The armed T cells in turn induce B cells to proliferate and turn into plasma cells making antibodies specific to the infecting pathogen thereby completing the biochemical pathway to provide adaptive immunity. Germinal centers in the body become a site of interaction of B cells and antigen-specific T cells with T cells constituting about 10% of germinal center lymphocytes. Therefore, interaction is limited by biological design.

A class of non-ionizing radiation, millimeter waves (MMW) are electromagnetic fields (EMF) of extremely high frequencies (30-300 GHz) corresponding to wavelengths of 10-1 mm. With relatively low photon energy of 0.000124-0.00124 eV (1 eV=1.6 10-19 J), MMW do not affect inter-atomic bonds but excite rotational, torsional and vibration modes of molecules through resonance. Irradiation at a resonant frequency with the appropriate intensity gives rise to biological effects of changes in organelle structures and cell membranes [4-6]. For example, irradiation with low power MMW at 42 GHz and power density <10 mW/cm² reduces cell proliferation in yeast [7] although irradiation with 341 GHz irradiation at 5.78 mW/cm² enhances yeast growth [8], the dichotomy attributed to the Frohlich hypothesis based on electromagnetic resonance. The observations suggest different frequencies and powers are capable of eliciting distinct cellular responses. In the case of humans, guidelines from the International Commission on Non-Ionizing Radiation Protection (ICNIRP) stipulate measurement of power density (PD) using units of W/m² for exposure of biological tissue to MMW irradiation. The ICNIRP recommends limiting power density to 200 W/m² in order to limit adverse thermal effects on biological tissue. MMW irradiation within 70-300 GHz range over a long-term duration (94 hours) maintained at a low incident power of 10 μW so as not to adversely increase cell temperature (non-thermal effects) [9] reported no difference in cellular activity or exposure derived cytotoxicity between exposed and sham normal human skin fibroblast (NB1RBG) or human glioblastoma (A172) cells.

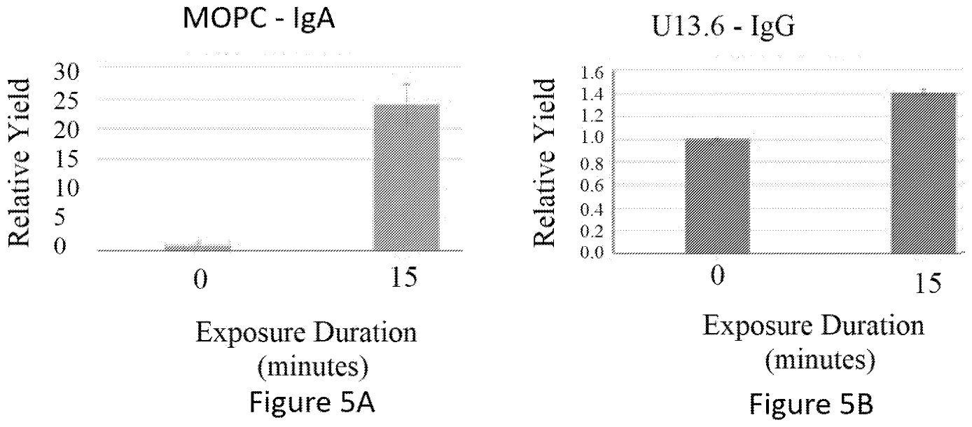

To determine the effect of MMW exposure on antibody production from murine B hybridoma cells, the antibody yields from 2 different cell lines were tested. Those tests preliminary indicate that in some embodiments, ideal irradiation time is 15 minutes at room temperature. IgA antibody yield from MOPC315.BM cells was enhanced by a factor of 24.05±3.32 as compared to control (FIG. 5A). IgG antibody yield from U13.6 cells was enhanced by a factor of 1.41±0.03 relative to control (FIG. 5B). The difference in yield enhancement as normalized to control for the two different cell lines subjected to the same exposure conditions indicates that the response to MMW stimulation is typically cell clone dependent.

In other studies, Lawler and colleagues [23] reported that at a power density ≥26 W/m² discontinuous exposure of human fibroblasts in a repeated interval-spaced radiation regime at 60 GHz was sufficient to stimulate: (a) formation of genomic secondary structures including G-quadruplex and i-motifs altering DNA structural dynamics without causing DNA damage; and (b) non-thermal transcriptome alterations enhancing protein synthesis. Higher power density (40 W/m²) repeated interval-spaced exposure spanning 30-40 GHz of chondrocytes demonstrated that MMW exposure non-thermally stimulated protein synthesis by regulating the voltage-gated K+ channel reversibly [24]. These observations suggest that MMW non-thermal effects (when exposure power is below the ICNIRP limits) are frequency of radiation dependent besides power dose dependency as discussed above. Further, under these conditions the effects are reversible, and transient being terminated with the end of radiation (see Guidelines for limiting exposure to electromagnetic fields (100 KHz to 300 GHz) 2020).

Similarly, the stimulated enhancement of antibody production resulting from the herein-disclosed frequency-swept MMW excitation regime which also involved repeating parameters of frequency skipping (see Materials and Methods), might be explained by several mechanisms including: (a) the formation of genomic secondary structures affecting transcription; and (b) radiation induced voltage changes on the cell membrane promoting exocytosis among others; as suggested from the evidential reports compared above. These effects during electromagnetic stimulation leads the system to undertake enhanced protein synthesis [24]. The observations further indicate that this switch ON continues until the eventual depletion of the culture medium or the termination of radiation switches OFF the system; whichever occurs earlier and hence determines the rate limiting step of this effect.

Delivery of MMW Radiation to Enhance Antibody Production in Hybridoma Cells does not Induce Toxicity

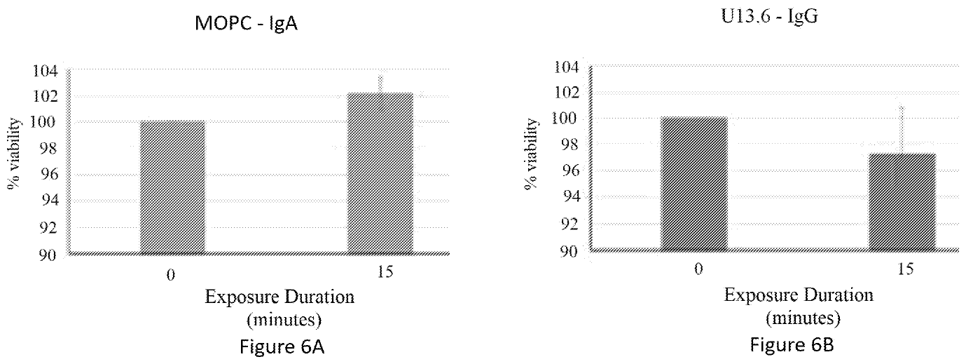

Experiments performed by the Inventors involved MMW radiation at power densities less than the ICNIRP stipulated safety standard of 200 W/m² dose. Viability measurements of both the MOPC315.BM and U13.6 cell lines subjected to MMW treatment induced no cytotoxic affect (FIGS. 6A and 6B), an observation consistent with a previous demonstration that long-term exposure at 10 W/m² over 24 hours at 40 GHz does not cause genotoxicity nor heat shock protein expression in either HCE-T and SRA01/04 human epithelial cells [25].

On the higher limits of cellular exposure, MMW irradiation at 60 GHz and an Incident Power Density (IPD) of 20 mW/cm² corresponding to the ICNIRP safe public exposure standards caused thermal stress on human skin cell lines [10]. However, within an IPD range of 5-15 mW/cm² irradiation with frequencies of 40-60 GHz stimulate hypoalgesic and immune effects [11]. Further, reduction of IPD to 0-5 mW/cm² eliminates generation of cellular stress or thermal effects [11]. Additional reports indicate healthy human epithelial cells tolerate irradiation at 60 GHz and 1 mW/cm² without developing effects of cytotoxicity. The Inventors experiments with MMW within 75-105 GHz at 0.2 mW/cm² led to stable in vitro temperature range of 24-26° C. measured over exposures lasting up to 4 minutes. Interestingly, MMW exposure at 42 GHz and 31.5 mW/cm² delivered on nose tip of mice for 30 minutes/day (over 3 days) led to enhanced activation, proliferation, and effector functions of T-cells [14].

In the adaptive immune response activated/armed T-cells respond in two different ways generating distinct types of effector cells based on the cytokine profile they produce [15]. The Th1 type of CD4 T cells secrete IFN-g and TNF-α cytokines associated with inflammation and induction of cell mediated immune response [16]. The Th2 type produces IL-4, IL-5, and IL-10 cytokines helping B cells to proliferate and differentiate into antibody production powerhouses. In the 42 GHz experiment reported above [14], observations suggested MMW exposure activated the Th1 type of immune response. Further, experiments involving MMW exposure at 61 GHz and 31.5 mW/cm² delivered on nose tip of mice for 30 minutes/day (over 3 days) demonstrated T-cell proliferation and activation due to the presence of macrophages actively functioning as APCs [17].

Due to the resonance involved in eliciting biological responses from MMW exposure as described above; different frequency range and power dosages can result in T-cell activation linked B-cell induction to produce antibodies. Further, Th1 and Th2 type responses are also cross-regulated in a reciprocal manner through IFN-g inhibiting Th2 cells and IL-10 inhibiting Th1 cells [18-20].

The invention is related to irradiation of immune cells, specifically using a millimeter wave radiation source or a terahertz radiation source to irradiate immune cells having a frequency of between about 70 and about 110 GHz in the presence of an antigen, which leads to an increase in the rate of antibody production. In the experiments, the increase in antibody production was estimated using optical density measurements.

### Materials and Methods

### Cell Culture

Murine MOPC315.BM plasmacytoma cells (Yado, S., Luboshits, G., Hazan, O., Or, R., & Firer, M. A. (2019)). Long-term survival without graft-versus-host-disease following infusion of allogeneic myeloma-specific VB T cell families. Journal for immunotherapy of cancer, 7(1), 301. https://doi.org/10.1186/s40425-019-0776-9), which produce a mAb of IgA isotype, were cultured at 37° C. in 5% CO₂ in RPMI 1640 (Sigma-Aldrich, Rehovot, Israel) supplemented with 10% FBS, 1% MEM NEAA 100× (Gibco), 0.005% 1 M I-thioglycerol, 0.03% Gensumycin 40 mg/ml (Sigma-Aldrich) and 2 mM L-glutamine (Biological Industries, Beit Haemek, Israel). U13.6 cells (Firer, M. A., Laptev, R., Kasatkin, I., & Trombka, D. (2003)). Specific destruction of hybridoma cells by antigen-toxin conjugates demonstrate an efficient strategy for targeted drug therapy in leukemias of the B cell lineage. (Leukemia & lymphoma 44(4), 681-689. https://doi.org/10.1080/1042819031000063381), a mouse hybridoma which produces a mAb of IgG isotype, were grown in DMEM containing 15% HS, 2 mML-glutamine, and combined antibiotics.

### Millimeter Wave Treatment of Hybridoma Cells

Cells were seeded at 1×10⁵ cells/ml in 200 microliter in Eppendorf tubes and exposed to MMW treatment for 15 minutes. Frequency Swept MMW radiation (85-110 GHZ, 200 steps, 5 dBm) was generated using a signal generator (N5183B, 9 kHz-20 GHz, Keysight Technology, Santa Rosa, CA, USA) and 6×active frequency multiplier (QMM-311220025, Quinstar Tech Inc., Torrance, CA, USA). An open-ended waveguide (QWH-WPRROO, Quinstar Tech Inc., Torrance, CA, USA) was used for MMW emission at 141.5±25.5 W/m². Power density measurements were calibrated using a digital storage oscilloscope (DSO-X 2004A, Agilent Technology, Santa Clara, CA, USA). Following treatment, the cells were cultured for a further 48 hours. Cell viability was determined using the XTT-based Cell Proliferation assay after which the samples were centrifuged at 400×g for 15 minutes and the supernatant used to antibody titer by ELISA (see below).

### Antibody Titer Determination

An ELISA was used to measure anti-Dinitrophenol (DNP) IgG antibodies from U13.6 cells, 96-well microplates (Nunc, Maxisorb) were coated overnight at 40 C° with a 5 μg/ml solution of DNP-BSA in PBS. The wells were washed with 0.1% Tween-20/PBS and then blocked for 30 minutes at room temperature (RT) with 1% BSA/0.02% NaN₃. After washing, culture supernatants diluted ¼, ⅛, 1/16, 1/32 or 1/64 in culture medium were added to appropriate wells for 2 hours at RT. As a positive control, a previously calibrated culture supernatant was used, while culture medium was used as a negative control. The wells were again washed and probed with horseradish peroxidase (HRP)-labeled goat anti-mouse IgG secondary antibody (Southern Biotech) for an hour at RT. The wells were washed and TMB substrate solution (Merck) was added to the wells, the plate was incubated for 10 mins in the dark and the enzymatic reaction was stopped by addition of 3M sulfuric acid (Sigma-Aldrich). Absorbances were then read at 635 and 450 nm on an Infinite m200 spectrophotometer (Tecan).

To measure IgA production from MOPC315.BM cells, the ELISA procedure above was modified as follows: microplate wells were coated with 5 μg/ml goat anti-mouse IgA (Thermo-Fischer) overnight at 40 C°. After incubation with samples, wells were probed with goat anti-mouse IgA FC-HRP (Southern Biotech). All the results were normalized to non-irradiated, positive control Ab-containing supernatants.

The experimental results show an increase of antibodies without being detrimental to cell viability.

### Irradiation Parameters

1. Cell concentration: 1 × 10⁵ cells/ml
2. Viability measurement indicates % live cells from start of experiment
3. 200 microliters of (cells + media) per well in 96-well plate
4. Sweeping Frequency (85-110 GHz)

| | |
|---|---|
| Points/Steps: 200; | Power: 5 dBm (constant) |

5. Oscilloscope readings
Power range before insertion

| | |
|---|---|
| 540-360 mV (outgoing); | 35-19 mV (reflected) |

Power range after insertion

| | |
|---|---|
| 540-360 mV (outgoing); | 50-20 mV (reflected) |

```
6. The aperture of the MMW source was directed into a waveguide.
  The waveguide was placed 3 mm above the surface of the 96-well plate
  surface.
```

### Calculation

### From Graph and Table

Range of power delivered: 2-1.4 mW (outgoing)=(0.002-0.0014) W
Cross sectional area of waveguide: 3 mm×4 mm=12 mm²=0.12 cm²=0.000012 m²

### Power Density:

$$( 2 - 1.4 ) mW / 0.12 {cm}^{2} = ( 16.67 - 11.67 ) mW / {cm}^{2}$$

$$( 0.002 - 0.0014 ) W / 0.000012 {m}^{2} = ( 166.67 - 116.67 ) W / {m}^{2}$$

### Example 1: MMW Radiation Enhances Antibody Production in Two Different Ig Class Platforms

Cells were seeded at 1×10⁵ cells/ml in 200 microliters of cells and media per well in a 96-well plate and delivered 15 minutes of MMW treatment. Post-treatment samples were incubated for 48 hours under physiological conditions and the cell media used for antibody titration.

At the end of the 48-hour incubation period absorbance of known antibody concentrations at 450 nm was measured spectrophotometrically to generate standard concentration curves for yield measurement. Using this plot, an equation of exponential fit was generated. Optical density readings of the wells were then used to calculate their corresponding antibody titers using the equation generated.

Results are shown in FIGS. 5A and 5B, where each column in the graphs represents the average and standard deviation of two readings.

It is seen that delivery of MMW radiation enhances IgA antibody production in MOPC cells by a factor of 24.05±3.32 while IgG antibody production in U13.6 cells was enhanced by a factor of 1.41±0.03.

### Example 2: MMW Radiation that Enhances Antibody Production in Two Different Ig Class Platforms does not Adversely Effect Cell Viability

Cells were seeded at 1×10⁵ cells/ml in 200 microliters of cells and media per well in a 96-well plate and delivered 15 minutes of MMW treatment. Post-treatment samples were incubated for 48 hours under physiological conditions and the cell media used for antibody titration.

At the end of the incubation periods, appropriate XTT kits were done to measure viability and absorbance at 450 nm was measured spectrophotometrically according to the manufacturer's protocol. % viability was calculated as (Optical Density of MMW in a treated well)/(Optical Density of MMW in untreated well)×100.

All % viability values were normalized by taking the value of “0 minutes exposure” as 100%.

Results are shown in FIGS. 6A and 6B, where each column in the graphs represents the average and standard deviation of two readings.

It is seen that delivery of MMW radiation to elicit enhanced antibody production does not induce toxicity in either MOPC or U13.6 cells.

Unless otherwise defined, all technical and scientific terms used herein have the same meaning as commonly understood by one of ordinary skill in the art to which the invention pertains. In case of conflict, the specification, including definitions, takes precedence.

As used herein, the terms “comprising”, “including”, “having” and grammatical variants thereof are to be taken as specifying the stated features, integers, steps or components but do not preclude the addition of one or more additional features, integers, steps, components or groups thereof. These terms encompass the terms “consisting of” and “consisting essentially of”. As used herein, the indefinite articles “a” and “an” mean “at least one” or “one or more” unless the context clearly dictates otherwise.

As used herein, when a numerical value is preceded by the term “about”, the term “about” is intended to indicate +/−10%. As used herein, a phrase in the form “A and/or B” means a selection from the group consisting of (A), (B) or (A and B). As used herein, a phrase in the form “at least one of A, B and C” means a selection from the group consisting of (A), (B), (C), (A and B), (A and C), (B and C) or (A and B and C).

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the scope of the appended claims.

Citation or identification of any reference in this application shall not be construed as an admission that such reference is available as prior art to the invention.

Section headings are used herein to ease understanding of the specification and should not be construed as necessarily limiting.

### REFERENCES

1. Carvalho, L. S., Silva, O. B., Almeida, G. C., Oliveira, J. D., Parachin, N. S., & Carmo, T. S. (2017). Production processes for monoclonal antibodies. Fermentation Processes. doi: 10.5772/64263.
2. Antigen-presenting cells. (Accessed April 2020). Available from: https://www.nature.com/subjects/antigen-presenting-cells
3. Hong S, Zhang Z, Liu H, Tian M, Zhu X, Zhang Z, Wang W, Zhou X, Zhang F, Ge Q, Zhu B, Tang H, Hua Z, Hou B. B Cells Are the Dominant Antigen-Presenting Cells that Activate Naive CD4+ T Cells upon Immunization with a Virus-Derived Nanoparticle Antigen. Immunity. 2018 Oct. 16; 49 (4): 695-708.e4. doi: 10.1016/j.immuni. 2018.08.012. Epub 2018 Oct. 2.
4. Siegel P H, Pikov V. Impact of low intensity millimetre waves on cell functions. Electron Lett 2010, 46 (26), pp 70-72. DOI: http://www.thz.caltech.edu/siegelpapers/IET_Dec2010.pdf
5. Shapiro M G, Priest M F, Siegel P H, Bezanilla F. Thermal Mechanisms of Millimeter Wave Stimulation of Excitable Cells. Biophys J 2013, 104 (12), pp 2622-8. DOI: https://www.sciencedirect.com/science/article/pii/S0006349513005705?via % 3Dihub
6. Curecheriu L, Foca-Nici E, Vlahovici A L, Avadane O, Sandu D D, Creangai D, Miclaus S. Radiofrequency wave effects on DNA and RNA levels in some animal tissues. Rom. Journ Phys 2007, 52 (3-4), pp 389-395. DOI: http://www.nipne.ro/rjp/2007_52_3-4/0389_0395.pdf
7. Grundler W, Keilmann F. Sharp Resonances in Yeast Growth Prove Nonthermal Sensitivity to Microwaves. Phys. Rev. Lett. 1983, 5, pp 1214-1216. DOI: https://journals.aps.org/prl/abstract/10.1103/PhysRevLett.51.1214
8. Hadjiloucas S, Chahal M S, Bowen J W. Preliminary results on the non-thermal effects of 200-350 GHz radiation on the growth rate of S. cerevisiae cells in microcolonies. Phys Med Biol 2002, 47 (21), pp 3831-9. DOI: https://iopscience.iop.org/article/10.1088/0031-9155/47/21/322
9. Yaekashiwa N, Otsuki S, Hayashi S, Kawase K. Investigation of the non-thermal effects of exposing cells to 70-300 GHz irradiation using a widely tunable source. J Radiat Res 2018, 59 (2), pp 116-121. DOI: https://academic.oup.com/jrr/article/59/2/116/4769636
10. Le Quement C, Nicolaz C N, Habauzit D, Zhadobov M, Sauleau R, Le Dréan Y. Impact of 60-GHz millimeter waves and corresponding heat effect on endoplasmic reticulum stress sensor gene expression. Bioelectromagnetics. 2014, 35 (6), pp 444-51. DOI: 10.1002/bem.21864. Epub 2014 Aug. 6.
11. Zhadobov M, Chahat N, Sauleau R, Le Quement C, Le Drean Y. Millimeter-wave interactions with the human body: State of knowledge and recent advances. International Journal of Microwave and Wireless Technologies 2011, 3 (2), pp 237-247. DOI: https://doi.org/10.1017/S1759078711000122
12. Koyama S, Narita E, Shimizu Y, Suzuki Y, Shiina T, Taki M, Shinohara N, Miyakoshi J. Effects of Long-Term Exposure to 60 GHz Millimeter-Wavelength Radiation on the Genotoxicity and Heat Shock Protein (Hsp) Expression of Cells Derived from Human Eye. Int J Environ Res Public Health. 2016, 13 (8), pp 802. DOI: https://www.ncbi.nlm.nih.gov/pmc/articles/PMC4997488/13.
13. International Telecommunication Union. RECOMMENDATION ITU-R P.676-10 Attenuation by atmospheric gases. Available from: https://www.itu.int/dms_pubrec/itu-r/rec/p/R-REC-P.676-10-201309-S!! PDF-E.pdf
14. Makar V, Logani M, Szabo, Ziskin M. Effect of millimeter waves on cyclophosphamide induced suppression of T cell functions. Bioelectromagnetics. 2003 July; 24 (5): 356-65. doi: https://www.ncbi.nlm.nih.gov/pubmed/12820293
15. Abbas A K, Murphy K M, Sher A. Functional diversity of helper T lymphocytes.Nature. 1996 Oct. 31; 383 (6603): 787-93. doi: https://www.ncbi.nlm.nih.gov/pubmed/8893001
16. Fearon D T, Locksley R M. The instructive role of innate immunity in the acquired immune response. Science. 1996 Apr. 5; 272 (5258): 50-3. doi https://www.ncbi.nlm.nih.gov/pubmed/8600536
17. Makar V R, Logani M K, Bhanushali A, Alekseev S, Ziskin M C. Effect of cyclophosphamide and 61.22 GHz millimeter waves on T-cell, B-cell, and macrophage functions. Bioelectromagnetics. 2006 September; 27 (6): 458-66. doi: https://www.ncbi.nlm.nih.gov/pubmed/16622862
18. Fiorentino D F, Bond M W, Mosmann T R. Two types of mouse T helper cell. I V. Th2 clones secrete a factor that inhibits cytokine production by Th1 clones. J Exp Med. 1989 Dec. 1; 170 (6): 2081-95. doi: https://www.ncbi.nlm.nih.gov/pubmed/2531194
19. Constant S L, Bottomly K. Induction of Th1 and Th2 CD4+ T cell responses: the alternative approaches. Annu Rev Immunol. 1997; 15:297-322. doi: https://www.ncbi.nlm.nih.gov/pubmed/9143690
20. Matar P, Rozados V R, Gervasoni S I, Scharovsky G O. 2001. Down regulation of T-cell-derived IL-10 production by low-dose cyclophosphamide treatment in tumor-bearing rats restores in vitro normal lymphoproliferative response. Int Immunopharmacol 1 (2): 307-319. doi: https://www.ncbi.nlm.nih.gov/pubmed/11360931
21. Matar P, Rozados V R, Gervasoni S I, Scharovsky G O. 2002. Th2/Th1 switch induced by a single low dose of cyclophosphamide in a rat metastatic lymphoma model. Cancer Immunol Immunother 50 (11): 588-596. doi: https://www.ncbi.nlm.nih.gov/pubmed/11807622
22. Mehta-Damani A, Markowicz S, Engleman E G. Generation of antigen-specific CD4+ T cell lines from naive precursors. Eur J Immunol. 1995 May; 25 (5): 1206-11. doi: https://www.ncbi.nlm.nih.gov/pubmed/7774624
23. Lawler et al., in Biomedical optics express (2022), 13 (5), 3131-3144. https://doi.org/10.1364/BOE.458478
24. Li et al., Journal of bone and mineral metabolism (2014), 32 (4), 367-377. https://doi.org/10.1007/s00774-013-0513-2)
25. Koyama et al in Journal of radiation research (2019), 60 (4), 417-423. https://doi.org/10.1093/jrr/rrz017.

1-27. (canceled)

28. A method for producing monoclonal antibodies, comprising:
i. providing antibody-producing cells;
ii. incubating said cells in vitro in the presence of an antigen for an incubation period under conditions suitable for said cells to produce antibodies; and
iii. during, before or after at least some of said incubation period, irradiating said cells with millimeter wave (MMW) and/or terahertz radiation for an irradiation period;
thereby said cells producing antibodies.

29. The method of claim 28, wherein said antibody-producing cells are selected from the group consisting of: IgA-producing cells, IgG-producing cells, CHO cells, HEK cells and hybridoma cells.

30. The method of claim 28, wherein said antibody-producing cells are immune cells.

31. The method of claim 28, wherein at least some of said irradiation period is during said incubation period.

32. The method of claim 28, wherein all of said irradiation period is prior to said incubation period.

33. The method of claim 28, wherein a duration of said irradiation period is not less than about 5 minutes.

34. The method of claim 28, wherein a duration of said irradiation period is not less than about 10 minutes.

35. The method of claim 28 wherein said radiation comprises terahertz radiation having one or more frequencies of between about 0.1 THz (100 GHz) and about 10 THz (10000 GHz).

36. The method of claim 28, wherein said radiation comprises MMW radiation having one or more frequencies of between about 30 GHz and about 300 GHz.

37. The method of claim 28, wherein during at least some of said irradiation period, said irradiating is with a single frequency of radiation.

38. The method of claim 37, wherein during said irradiation period, the frequency of said single frequency changes.

39. The method of claim 38, said irradiation comprises frequency sweeping during at least some of the irradiation period so that at any given moment said cells are irradiated with only a single frequency but the specific single frequency of said single frequency that irradiates the cells at any given moment changes monotonously during said irradiation period.

40. The method of claim 28, wherein during at least some of said irradiation period, said irradiating is with multiple frequencies of radiation simultaneously.

41. The method of claim 28, wherein during said irradiation period, said irradiating has an average intensity of between about 10 W/m² and about 600 W/m².

42. The method of claim 28, wherein during said irradiation period, said irradiating has an average intensity of between about 50 W/m² and about 400 W/m².

43. The method of claim 28, wherein during said irradiation period, said irradiating has an average intensity of between about 100 W/m² and about 200 W/m².

44. The method of claim 28, wherein during said irradiation period, the intensity of said irradiating is within about 33% of the average intensity value.

45. The method of claim 28, wherein said irradiation period is a single contiguous period of time.

46. The method of claim 28, wherein said irradiation period comprises at least two time-segments, any two time segments separated by a rest period, wherein a sum of the durations of all said time segments is the duration of said irradiation period.

47. A device suitable for producing antibodies comprising:
an incubation module components suitable for incubation of antibody-producing cells for an incubation period therein; and
an irradiation module for irradiation of antibody-producing cells that are being incubated in said incubation module for an irradiation period with MMW and/or terahertz radiation.
